(19) United States
(12) Patent Application Publication
(10) Pub. No.: US 2008/0027292 A1
(43) Pub. Date: Jan. 31, 2008

(54) Computer-Implemented Method and Apparatus for Diabetes Management

(76) Inventors: Paul M. Rosman, Lyndhurst, OH (US); Scott M. Pappada, Warren, OH (US)

Correspondence Address: HOWARD M COHN;PATENT ATTORNEY LLC, 21625 CHAGRIN BLVD #220, CLEVELAND, OH, 44122, US

(21) Appl. No.: 11/868,660

(22) Filed: Oct. 8, 2007

(30) Foreign Application Priority Data
Apr. 10, 2006 (US) PCT/US06/13114

(51) Int. Cl. G06F 15/18; A61B 5/00

(52) U.S. Cl. 600/300; 706/21

(57) ABSTRACT

A computer-implemented method and apparatus assists a user with diabetes management. The apparatus and method enables the user to record a plurality of their life events; record a plurality of their emotions, each corresponding to one of the plurality of life events; record a plurality of their blood glucose levels, each corresponding to one of the plurality of life events; and predict their glucose changes corresponding to their engaging in one of the recorded life events, and having the corresponding emotion to prevent hypoglycemia.

### CROSS-REFERENCE TO RELATED APPLICATIONS

This application claims the benefit of U.S. Provisional Patent Application No. 60/669,431 filed on Apr. 8, 2005 which is incorporated in its entirety herein by reference.

This application also claims the benefit of International Application No. PCT/US2006/013114 filed on Apr. 10, 2006 which is incorporated in its entirety herein by reference.

### FIELD OF THE INVENTION

The present invention relates to management of diabetes and more particularly to a method and apparatus for assisting a user with diabetes management.

### BACKGROUND

Diabetes Mcllitus is a disease known since ancient times. In 1865 Bernard identified that “something” controlled glucose levels in the blood and that diabetes mellitus occurred because that “something” was deficient or missing. Later, insulin was discovered to be that “something” and it was given to people whereby diabetes mellitus became a possibly survivable disease.

Ever since insulin was first given to people, it has been characterized on a molecular level and the physiology of the human glucose-insulin action has been defined. This characterization of molecular and physiologic aspects of human glucose control includes the timing of glucose excursions and of the changes in the relationship between insulin action and glucose responsiveness during a 24-hour day.

As the pathophysiology of diabetes mellitus was studied and better defined, the technology of diabetes management was developed to allow for glucose measurement and insulin delivery to diabetic individuals throughout the day. Current clinical glucose measurement is primarily episodic, with single measurements of blood glucose taken with a finger stick and done by the diabetic individuals or by others.

Avoiding dysglycemia (low and high glucose levels) is vital to the clinical management of diabetes mellitus, and in many patients, current approaches are unsuccessful as measured by the occurrence of acute and chronic complication, and by the immense cost of diabetes care in this country and throughout the world. Low blood glucose levels are potentially devastating because they can produce coma and lesser degrees of brain dysfunction that can result in injury or death.

Low blood glucose levels may be unrecognized by people who have diabetes mellitus for several years, thereby causing an added danger. Furthermore, low blood glucose levels are the major impediment to clinically acceptable glucose level control in insulin dependent, diabetes mellitus patients.

High blood glucose levels are associated with increased risk of devastating long term complications in all people with diabetes mellitus. These complications include microvascular and macrovascular problems. Microvascular complications of diabetes mellitus include retinopathy (visual loss), nephropathy (renal or kidney failure) and neuropathy (loss of feeling, altered sensation, severe pain, or inability to recognize low blood glucose levels). Macrovascular complications of diabetes mellitus include myocardial infarction, increased cardiac death, and stroke. All of these complications are reduced by improved blood glucose control and many are reversible over time if glucose levels are normalized.

Current treatment, based upon episodic, patient obtained, finger stick glucose measurements, are not adequate to obtain clinical diabetes management targets because blood glucose levels can fall by 50% in 20 minutes, or increase by 200% in 15 minutes, depending upon the circumstances. Further, significant changes in glucose levels occur when patients are sleeping.

Diabetes Mellitus makes people's life more complicated. It can be difficult for a person to maintain a useful glucose diary, and it can be challenging to achieve “acceptable” glucose levels.

There is a critical need for people with Diabetes Mellitus to manage their specific glucose levels as well as individual factors that determine dysglycemia in people with diabetes mellitus. Further, there is a need to be able to identify and alert diabetic patients to life events and associated glucose trends that produce recurrent dysglycemia.

### ASPECTS AND SUMMARY OF INVENTION

An aspect of the invention is to assist people in managing their diabetes more successfully.

Another aspect of the invention is the use of a simple, intuitive approach to alert people to how their normal activities and feelings can affect their glucose levels and their diabetes management.

Yet another aspect of the invention is to allow a user to gain better control and successful management of their diabetes.

Still another aspect of the invention is to facilitate the recording of glucose levels, calories, carbohydrates, and insulin dosages.

Another aspect of the invention is to guide the user in documenting life activities such as sleep, meals and work, as well as emotions such as happiness, fatigue, and stress. The invention shows the user how these life events or activities may be related to changes in the user's glucose levels throughout the day and thereby enables the user to better manage their diabetes.

Still another aspect of the invention is to simplify the user's daily diabetes-related decisions so as to make diabetes management a more effective part of the user's life.

According to the present invention, a computer-implemented method assists a user with diabetes management. The method comprises the steps of recording a plurality of life events experienced by the user; recording a plurality of emotions of the user, each corresponding to one of the plurality of events; recording a plurality of blood glucose levels of the user, each corresponding to one of the plurality of events; and predicting glucose changes of the user corresponding to the user engaging in one of the recorded life events, and having the corresponding emotion so the user will be able to prevent hypoglycemia.

Further according to the present invention, the method comprises the steps of recording a plurality of insulin dosages, each corresponding to one of the plurality of life events; and predicting the glucose level of a user corresponding to the user engaging in one of the recorded life events, having the corresponding emotion, and the corresponding recorded insulin dosage so the user will be able to prevent hypoglycemia.

Still further according to the present invention, the method comprises the steps of recording grains of carbohydrates consumed, each corresponding to one of the plurality of life events; and predicting the glucose level of a user corresponding to the user engaging in one of the recorded events, having the corresponding emotion, the corresponding recorded insulin dosage, and the corresponding grams of carbohydrates consumed so the user will be able to prevent hypoglycemia.

Also according to the present invention, the plurality of emotions of the user includes happiness, pain, fatigue, fear, worry and stress and the plurality of life events experienced by the user include sleep, meals, work, exercise, play, and study.

Moreover according to the present invention, the hypoglycemia is a high glucose level or a low glucose level.

According to the present invention, the method includes the step of creating a chart with the plurality of life events; the plurality of emotions, each corresponding to one of the plurality of life events; and the plurality of blood glucose levels, each corresponding to one of the plurality of life events.

Also according to the present invention the method includes the step of creating a chart with plurality of life events; the plurality of emotions, each corresponding to one of the plurality of events; the plurality of blood glucose levels, each corresponding to one of the plurality of events; the grains of carbohydrates consumed, each corresponding to one of the plurality of life events; the plurality of insulin dosages, each corresponding to one of the plurality of life events; and the corresponding grams of carbohydrates consumed, each corresponding to one of the plurality of life events; so the user will be able to prevent hypoglycemia.

According to the present invention, a computer-implemented apparatus for assisting a user with diabetes management is characterized by structure for recording a plurality of life events experienced by the user; structure for recording a plurality of emotions of the user, each corresponding to one of the plurality of events; structure for recording a plurality of blood glucose levels of the user, each corresponding to one of the plurality of events; and structure for predicting glucose changes of the user corresponding to the user engaging in one of the recorded events, and having the corresponding emotion so the user will be able to prevent hypoglycemia.

Also according to the present invention, the computer-implemented apparatus is further characterized by structure for recording a plurality of insulin dosages, each corresponding to one of the plurality of events; and structure for predicting the glucose level of a user corresponding to the user engaging in one of the recorded events, having the corresponding emotion, and the corresponding recorded insulin dosage so the user will be able to prevent hypoglycemia.

Still further according to the present invention, the computer-implemented apparatus is further characterized by structure for recording grams of carbohydrates consumed, each corresponding to one of the plurality of events; and structure for predicting the glucose level of a user corresponding to the user engaging in one of the recorded life events, having the corresponding emotion, the corresponding recorded insulin dosage, and the corresponding grams of carbohydrates consumed so the user will be able to prevent hypoglycemia.

Moreover according to the present invention, the computer-implemented apparatus is further characterized in that the plurality of emotions of the user include happiness, pain, fatigue, fear, worry and stress and the plurality of life events experienced by the user include sleep, meals, work, exercise, play, and study.

According to the present invention, the hypoglycemia is a high glucose level or a low glucose level.

Still further according to the present invention, the computer-implemented apparatus is further characterized by structure for creating a chart with the plurality of life events; the plurality of emotions, each corresponding to one of the plurality of life events; and the plurality of blood glucose levels, each corresponding to one of the plurality of life events.

Further according to the present invention, the computer-implemented apparatus is further characterized by structure for creating a chart with the plurality of life events; the plurality of emotions, each corresponding to one of the plurality of events; the plurality of blood glucose levels, each corresponding to one of the plurality of events; the grams of carbohydrates consumed, each corresponding to one of the plurality of life events; the plurality of insulin dosages, each corresponding to one of the plurality of life events; and the corresponding grams of carbohydrates consumed, each corresponding to one of the plurality of life events; so the user will be able to prevent hypoglycemia.

### BRIEF DESCRIPTIONS OF THE FIGURES

The structure, operation, and advantages of the present invention will become further apparent upon consideration of the following description taken in conjunction with the accompanying figures (Figs.). The figures are intended to be illustrative, not limiting.

Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, often both reference numerals and legends (labels, text descriptions) may be used to identify elements. If legends are provided, they are intended merely as an aid to the reader, and should not in any way be interpreted as limiting.

FIG. 1 is a plan view of a personal digital assistant (PDA) such as a Pocket PC showing the main menu of a diabetes management program, according to the present invention;

FIG. 2 is a plan view of a Pocket PC PDA showing the menu for selecting an event for inputting into the diabetes management program, according to the present invention;

FIG. 3 is a plan view of a Pocket PC PDA showing the menu for selecting an emotion for inputting into the diabetes management program, according to the present invention;

FIG. 4 is a plan view of a Pocket PC PDA showing the menu for selecting an event for a BG value into the diabetes management program, according to the present invention;

FIG. 5 is a plan view of a Pocket PC PDA showing the menu for inputting the insulin dosage into the diabetes management program, according to the present invention; and

FIG. 6 is a plan view of a Pocket PC PDA showing the menu for inputting the number of grams of carbohydrates consumed into diabetes management program, according to the present invention.

### DETAILED DESCRIPTION OF THE PREFERRED EMBODIMENT

The invention relates to a computer-implemented diabetes management program, comprising diabetes management software for achieving the objectives and purposes set forth herein. The computer-implemented diabetes management program can be and is described as being implemented on a personal digital assistant (PDA) device, such as a Pocket PC. However, it is within the scope of the invention to implement the computer-implemented diabetes management program on a desktop Personal Computer (PC).

Installing/Accessing the Diabetes Management Program

The diabetes management program is first installed on a computer such as a desktop PC (not shown). Then the diabetes management program is transferred to a Pocket PC PDA 10. This can be accomplished by initially connecting the desktop PC with the installed diabetes management program to the Pocket PC PDA 10 via a program such as for example, Microsoft ActiveSync. Then, the Explore icon on the My Pocket PC window of the Pocket PC PDA 10 is clicked. Next, the file with the diabetes management program is dragged or copied from the desktop PC and pasted into the Pocket PC window of the PDA 10.

Once the diabetes management software program is installed on the PDA 10, the file can be accessed and installed in the conventional manner. For example:
(1) Click Start on the Pocket PC PDA
(2) Click on Programs
(3) From Programs Select File Explorer
(4) Click on the diabetes management software program file (this will install the program)
   Now the diabetes management software program can be accessed and run in the conventional manner.
   For example:
(1) Click Start in the upper left corner of the PDA screen.
(2) Click on Programs icon.
(3) Click on the diabetes management program icon.
   Using the Diabetes Management Software

In the description that follows, the invention is described largely in the context of a familiar user interface, such as the Microsoft Windows™ operating system and graphic user interface (GUI) environment. It should be understood that although certain operations, such as clicking on a button, selecting a group of items, drag-and-drop, and the like, may be described in the context of using a graphical input device, such as a mouse, it is within the scope of the invention that other suitable input devices, such as keyboard, voice or other audio input, optical or other video input, tablets, and the like, could alternatively be used to perform the described functions. Also, where certain items are described as being highlighted or marked, so as to be visually distinctive from other (typically similar) items in the graphical interface, that any suitable structure of highlighting or identifying or marking the items visually, audibly or otherwise can be employed, and that any and all such alternatives are within the intended scope of the invention.

The Main Menu

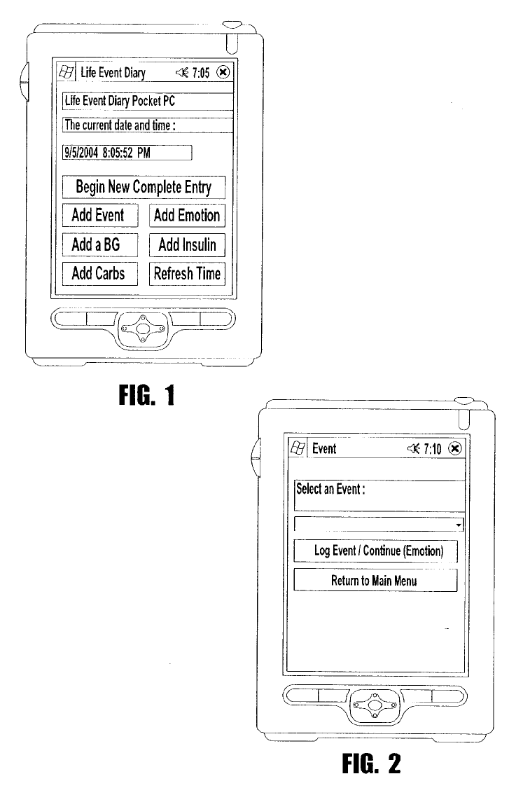

After the user opens the diabetes management program, the main menu 12 will be visible, as shown in FIG. 1.

From this window, the user is automatically given the date, and time and a variety of options for diary entries. By clicking on the Begin New (Complete Entry button 14, the user will be prompted to enter life events, emotions, blood glucose (BG) values, insulin dosages, and carbohydrates consumed. If the user does not wish to enter all or most of these, they can forego clicking the Begin New Complete Entry button 14 and instead specify what they would like to enter via clicking one of the other buttons to log only specific things.

It is, however, preferable that a user start with the “Begin New Complete Entry” 14 button each time. Every time the user encounters the main screen 12, it is advisable to click the Refresh Time button 16 to ensure the time and date in the log book are complete and correct.

When an entry is completed, it is also preferable that the main window 12 is left open and the Pocket PC PDA 10 is turned off by pressing the power button 18 at the top of the Pocket PC PDA. By following these instructions, the Pocket PC PDA 10 should open to the Main Menu Window 12 each time it is opened. The user should always remember to click Refresh Time button 16 to be certain the date and time are correct before entering an entry.

Adding a Life Event

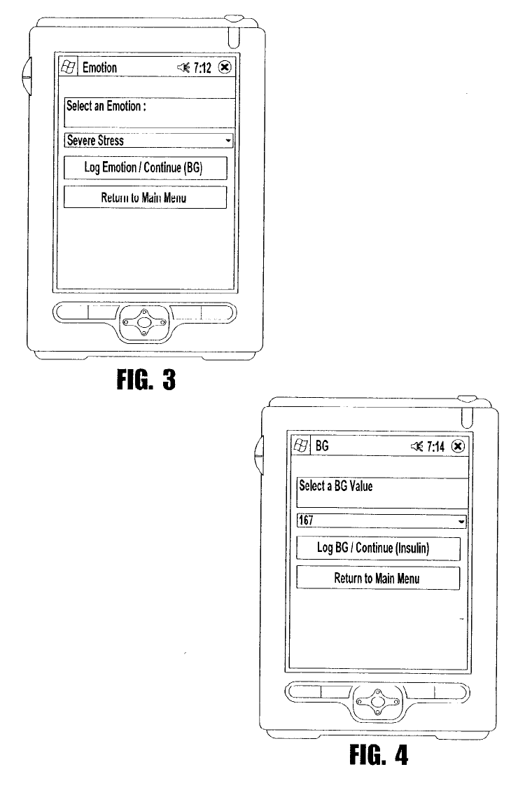

The user may want to log in a life event such as for example eating, exercising, working, and/or sleeping with other information, as discussed below. By clicking of the Add Event button 20 of PDA 10 shown in FIG. 1, the screen 22 shown in FIG. 2 will be shown on PDA 10. Then by clicking on button 24, a drop down menu will be displayed on the screen 22 and provide the user with a list of events from which to choose. The user can then click on one of the life events to select it. Then the user can either click the Return to Main Menu button 26 to go back to the Main Menu screen 12, as shown in FIG. 1, or click the Log Event/Continue (Emotion) button 28 to go to the screen 32 shown in FIG. 3 for logging in an emotion.

Adding an Emotion

If the user clicks the Log Event/Continue (Emotion) button 28 on the screen shown in FIG. 2 or the Add Emotion button 30 on screen 12 shown in FIG. 1, the screen 32 appears on PDA 10 (see FIG. 3). To log in an emotion, such as for example happiness, sadness, being worried, fear, or severe stress, the user clicks the Select an Emotion button 34 of PDA 10 shown in FIG. 3 and a drop down menu will be displayed on the screen 32 and provide the user with a list of emotions from which to choose the desired emotion. The user can then click on one of the emotions to select it and it will appear in box 38. Then the user can either click the Return to Main Menu button 40 to go back to the Main Menu screen 12, as shown in FIG. 1, or click the Log Event/Continue (BG) button 42 to go to the screen 46 shown in FIG. 4 for logging in the blood glucose values.

Adding a BG (Blood Glucose) Value

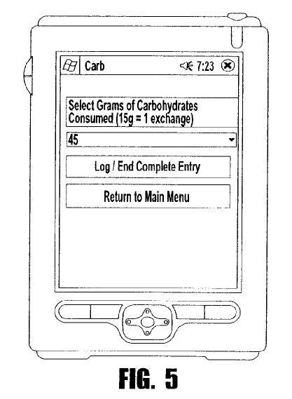

If the user clicks the Log Event/Continue (BG) button 42 on the screen shown in FIG. 3 or the Add Emotion button 44 on screen 12 shown in FIG. 1, the screen 46 appears on PDA 10 (see FIG. 4). To log in the Blood Glucose value, the user clicks the Select a BG Value button 48 of PDA 10. To increase the BG value click/hold on the up arrow 50 and to decrease the BG value click/hold on the down arrow 51. The selected BG value will appear in box 53. Then the user can either click the Return to Main Menu button 52 to go back to the Main Menu screen 12, as shown in FIG. 1, or click the Log BG/Continue (Insulin) button 54 to go to the screen 56 shown in FIG. 5 for logging in the Insulin Dosage.

Adding Units of Insulin

If the user clicks the Log Event/Continue (Insulin) button 54 on the screen shown in FIG. 4 or the Add Insulin button 58 on screen 12 shown in FIG. 1, the screen 56 appears on PDA 10, as shown in FIG. 5. From screen 56, the user is prompted to enter their insulin dosages. The first text box is for the whole units of insulin, whereas the second text box 62 is for entering tenths of a unit of insulin. To decrease the value of the insulin dosage, click/hold on the down arrow 64, 66 next to each text box 60, 62, respectively. To increase the value of the insulin dosage, click/hold on the up arrow 68,70 next to each text box 60, 62, respectively. After successfully entering a insulin dosage the user can return to the main menu 12 by clicking the Return to Main Menu button 72 or click the Log this/Continue(Carbs) button 74 and log the insulin dosage and continue to add carbohydrate intake.

Adding Grams of Carbohydrates Consumed

If the user clicks the Log this/Continue(Carbs) button 74 on the screen shown in FIG. 5 or the Add Carbs button 76 on screen 12 shown in FIG. 1, the screen 78 appears on PDA 10 in FIG. 6. The user can enter the approximate amount of carbohydrate intake at each meal or snack. The user can select the grams of carbohydrates consumed by clicking the Select Grams of (Carbohydrates Consumed (1.5 g=1 exchange) button 80 of PDA 10 shown in FIG. 6. To increase the Carbohydrates Consumed value, click/hold on the up arrow 82 and to decrease Carbohydrates Consumed value click/hold on the down arrow 84. The selected Carbohydrates Consumed value will appear in box 86. Then the user can either click the Return to Main Menu button 88 to go back to the Main Menu screen 12, as shown in FIG. 1, or click the Log/End Complete entry button 90.

Viewing the Selected Values Recorded in the Life Event Diary

The program creates a PC Microsoft Excel Spreadsheet as its main output file. To move this Excel Spreadsheet file onto the desktop PC:

1. Open ActiveSync and sync the Pocket PC PDA with your desk top PC via using the USB connection or USB, cradle if applicable

2. Click on the Explore Icon

3. Click in the drop down choice box in the tipper left corner where it says My Documents and select My Device

4. From here you can copy and paste or drag the LifeEventDiary.xls file onto your Desktop PC in any directory you wish.

5. Open the program to view a chart, for example as shown in Chart 1, showing the life events, emotions (mood), blood glucose (BG) values, hyposym, hypersym, insulin dosages, and carbohydrates for each reading.

CHART 1

| | | | | Blood | | | | |
|---|---|---|---|---|---|---|---|---|
| Date | Time | Life Event | Mood | Glucose | HypoSym | HyperSym | Insulin | Carbs |
| Jan. 12, 2005 | 5:20 PM | Dinner | Angry | 215 | N/A | Frequent | 15H 23L | 15 g |
| | | | | | | Urination | | pretzels |
| Aug. 23, 2004 | 17:42 | | | | | | | |
| Aug. 23, 2004 | 17:45 | | | 90 | | | 0.0 | |
| Aug. 23, 2004 | 18:25 | | | 126 | | | 1.1 | 30 |
| Aug. 23, 2004 | 18:26 | Dinner | | | | | | |
| Aug. 23, 2004 | 20:30 | | Fatigued | | | | 0.0 | |
| Aug. 23, 2004 | 20:31 | | | 64 | | | 0.0 | |
| Aug. 23, 2004 | 20:49 | | | | | | | 30 |
| Aug. 23, 2004 | 22:07 | | | | | | | |
| Aug. 23, 2004 | 22:08 | | | 125 | | | 0.5 | 15 |
| Aug. 24, 2004 | 12:15 | | | 99 | | | 1.0 | 45 |
| Aug. 24, 2004 | 16:02 | | | 266 | | | 1.5 | |
| Aug. 24, 2004 | 17:32 | | | 152 | | | 1.0 | 30 |
| Aug. 24, 2004 | 18:19 | | | | | | | |
| Aug. 24, 2004 | 18:19 | | | | | | | |
| Aug. 24, 2004 | 18:20 | Walking | Happy | | | | | |
| Aug. 24, 2004 | 20:27 | | | 56 | | | 0.0 | 30 |
| Aug. 24, 2004 | 20:56 | Snack | | | | | | |
| Aug. 24, 2004 | 20:57 | | Fatigued | | | | | |
| Aug. 24, 2004 | 22:03 | | | 79 | | | 0.0 | |
| Aug. 24, 2004 | 22:03 | Snack | | 40 | | | | |
| Aug. 24, 2004 | 22:04 | Sleep | | | | | | |
| Aug. 24, 2004 | 22:06 | Snack | | | | | | |
| Aug. 25, 2004 | 15:02 | | | | | | 0.0 | |
| Aug. 25, 2004 | 15:04 | | | 185 | | | | |
| Aug. 25, 2004 | 15:06 | Job@Work | Angry | 185 | | | | |
| Aug. 25, 2004 | 17:24 | Dinner | Fatigued | | | | | |
| Aug. 25, 2004 | 17:25 | | | 77 | | | | |
| Aug. 25, 2004 | 17:25 | | | | | | 1.0 | 45 |
| Aug. 25, 2004 | 21:08 | Snack | | | | | | |
| Aug. 25, 2004 | 21:09 | | | 187 | | | 1.0 | 15 |
| Aug. 25, 2004 | 21:10 | | Fatigued | | | | | |
| Aug. 26, 2004 | 6:08 | Breakfast | Happy | | | | | |
| Aug. 26, 2004 | 6:10 | | | | | | 1.0 | 30 |
| Aug. 26, 2004 | 6:10 | Arousal | | | | | | |
| | | (Waking up) | | | | | | |
| Aug. 26, 2004 | 9:22 | | | 102 | | | 0.5 | 15 |
| Aug. 26, 2004 | 9:23 | Snack | | | | | | |
| Aug. 26, 2004 | 11:03 | | | 92 | | | 0.0 | |
| Aug. 26, 2004 | 11:03 | | Fatigued | | | | | |
| Aug. 26, 2004 | 11:28 | | | 98 | | | | |
| Aug. 26, 2004 | 12:14 | | | 119 | | | 1.0 | 30 |
| Aug. 26, 2004 | 12:15 | Lunch | | | | | | |
| Aug. 26, 2004 | 13:44 | Snack | | | | | | |
| Aug. 26, 2004 | 15:07 | Job @ Work | | | | | | |
| Aug. 26, 2004 | 15:41 | | | 127 | | | | |
| Aug. 26, 2004 | 17:32 | | | | | | 1.0 | 45 |
| Aug. 26, 2004 | 17:32 | | | 69 | | | | |
| Aug. 26, 2004 | 17:42 | | | | | | 1.0 | |
| Aug. 26, 2004 | 20:34 | | | 61 | | | 0.0 | 30 |
| Aug. 26, 2004 | 20:36 | Inside House | Sick/ | 40 | | | | |
| | | | Nauseated | | | | | |
| Aug. 26, 2004 | 20:36 | | Sick/ | | | | | |
| | | | Nauseated | | | | | |
| Aug. 26, 2004 | 21:07 | | | 73 | | | 0.0 | 15 |

What does the User do with the Data from the Life Event Diary?

The user of the present invention has now written their own program for more successful management of their diabetes. The user is beginning to get information that they never had before. It is understood that this is just the beginning of collecting data for analysis. The reason is because if the program is used only once, its repetitiveness cannot be relied upon. If the program is used many times, the repetitiveness will enable the user to predict of glucose changes in certain situations. With this information, the user will be able to manage their glucose levels and diabetes more successfully.

Moreover, the data can be shown to a physician or nurse, particularly when discussing your diabetes management with them.

An advantage of the present invention is that it allows the user to rely on their own experiential knowledge intimacy of how their diabetes mellitus reacts to life events and the physiologic responsiveness for better control and successful management.

There is a clinical application of the computerized intensive life event diary programs with mathematical modeling of continuously generated glucose data that anticipates and alerts patients with diabetes mellitus to increased vulnerability to low and high glucose levels.

This system changes the paradigm of treatment in people with diabetes mellitus by making insulin delivery prospective based on the individuals life events and physiologic responsiveness, instead of being generalized or reactive to high or low glucose levels that have already occurred, as it is now.

This system has the potential to improve glucose control over time, thereby improving quality of life and clinical outcomes by avoiding acute and chronic complications of diabetes. Application of this system is designed to avoid the major impediment to effective diabetes control, namely hypoglycemia. By preventing hypoglycemia, this system will also prevent adverse effects from hypoglycemia unawareness as well as rebound hyperglycemia. It can recognize glycemic effects of exercise, sleep, or work in individuals.

This system is designed to prevent hyperglycemia as well. It will recognize meals or mealtimes associated with inadequate insulin use, as well as life events that require increased insulin doses such as emotional stress, pain, menses or arousal.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. A computer-implemented method of assisting a user with diabetes management comprising the steps of:
recording a plurality of life events experienced by the user;
recording a plurality of emotions of the user, each corresponding to one of the plurality of life events;
recording a plurality of blood glucose levels of the user, each corresponding to one of the plurality of life events; and
predicting glucose changes of the user corresponding to the user engaging in one of the recorded life events, and having the corresponding emotion so the user will be able to prevent hypoglycemia.

2. The method of claim 1, further comprising the steps of:
recording a plurality of insulin dosages, each corresponding to one of the plurality of life events; and
predicting the glucose level of a user corresponding to the user engaging in one of the recorded life events, having the corresponding emotion, and the corresponding recorded insulin dosage so the user will be able to prevent hypoglycemia.

3. The method of claim 2, further comprising the steps of:
recording grams of carbohydrates consumed, each corresponding to one of the plurality of life events; and
predicting the glucose level of a user corresponding to the user engaging in one of the recorded life events, having the corresponding emotion, the corresponding recorded insulin dosage, and the corresponding grams of carbohydrates consumed so the user will be able to prevent hypoglycemia.

4. The method of claim 3, wherein the plurality of emotions of the user include happiness, pain, fatigue, fear, worry and stress.

5. The method of claim 4, wherein the plurality of life events experienced by the user include sleep, meals, work, exercise, play, and study.

6. The method of claim 4, wherein the hypoglycemia is a high glucose level.

7. The method of claim 4, wherein the hypoglycemia is a low glucose level.

8. The method of claim 1, including the step of creating a chart with the plurality of life events; the plurality of emotions, each corresponding to one of the plurality of life events; and the plurality of blood glucose levels, each corresponding to one of the plurality of life events.

9. The method of claim 3 including the step of creating a chart with plurality of life events; the plurality of emotions, each corresponding to one of the plurality of events; the plurality of blood glucose levels, each corresponding to one of the plurality of events; the grams of carbohydrates consumed, each corresponding to one of the plurality of life events; the plurality of insulin dosages, each corresponding to one of the plurality of life events; and the corresponding grams of carbohydrates consumed, each corresponding to one of the plurality of life events; so the user will be able to prevent hypoglycemia.

10. A computer-implemented apparatus 10 for assisting a user with diabetes management characterized by:
means for recording a plurality of life events experienced by the user;
means for recording a plurality of emotions of the user, each corresponding to one of the plurality of life events;
means for recording a plurality of blood glucose levels of the user, each corresponding to one of the plurality of life events; and
means for predicting glucose changes of the user corresponding to the user engaging in one of the recorded life events, and having the corresponding emotion so the user will be able to prevent hypoglycemia.

11. The computer-implemented apparatus of claim 10, further characterized by:
means for recording a plurality of insulin dosages, each corresponding to one of the plurality of life events; and
means for predicting the glucose level of a user corresponding to the user engaging in one of the recorded life events, having the corresponding emotion, and the corresponding recorded insulin dosage so the user will be able to prevent hypoglycemia.

12. The computer-implemented apparatus of claim 11, further characterized by:
means for recording grams of carbohydrates consumed, each corresponding to one of the plurality of life events; and
means for predicting the glucose level of a user corresponding to the user engaging in one of the recorded life events, having the corresponding emotion, the corresponding recorded insulin dosage, and the corresponding grams of carbohydrates consumed so the user will be able to prevent hypoglycemia.

13. The computer-implemented apparatus of claim 12, further characterized in that the plurality of emotions of the user include happiness, pain, fatigue, fear, worry and stress.

14. The computer-implemented apparatus of claim 13, further characterized in that the plurality of life events experienced by the user include sleep, meals, work, exercise, play, and study.

15. The computer-implemented apparatus of claim 12, further characterized in that the hypoglycemia is a high glucose level.

16. The computer-implemented apparatus of claim 12, further characterized in that the hypoglycemia is a low glucose level.

17. The computer-implemented apparatus of claim 10, further characterized by:
means for creating a chart with the plurality of life events; the plurality of emotions, each corresponding to one of the plurality of life events; and the plurality of blood glucose levels, each corresponding to one of the plurality of life events.

18. The computer-implemented apparatus of claim 11, further characterized by:
means for creating a chart with the plurality of life events; the plurality of emotions, each corresponding to one of the plurality of life events; the plurality of blood glucose levels, each corresponding to one of the plurality of life events; the grams of carbohydrates consumed, each corresponding to one of the plurality of life events; the plurality of insulin dosages, each corresponding to one of the plurality of life events; and the corresponding grams of carbohydrates consumed, each corresponding to one of the plurality of life events; so the user will be able to prevent hypoglycemia.
